(19) United States
(12) Patent Application Publication
(10) Pub. No.: US 2008/0200672 A1
(43) Pub. Date: Aug. 21, 2008

(54) Highly enantioselective carbonyl reduction with borane catalyzed by chiral spiroborate esters derived from chiral beta-aminoalcohols

(76) Inventors: Margarita Ortiz-Marciales, Humacao, PR (US); Viatcheslav Stepanenko, Humacao, PR (US); Wildeliz Correa-Ramirez, Humacao, PR (US); Melvin de Jesus, Juncos, PR (US)

Correspondence Address: HOGLUND & PAMIAS, 256 ELEANOR ROOSEVELT STREET, SAN JUAN, PR, 00918, US

(21) Appl. No.: 11/512,599

(22) Filed: Aug. 30, 2006

Related U.S. Application Data
(60) Provisional application No. 60/712,669, filed on Aug. 30, 2005.

(51) Int. Cl. C07D 279/18; C07F 5/04; C07D 211/82

(52) U.S. Cl. 544/35; 546/339; 558/288

(57) ABSTRACT

Novel spiroborate esters derived from non-recemic 1,2-amino alcohols were examined as chiral catalyst in the borane reduction of acetophenone and other aromatic ketones at room temperature. The optically active alcohols were obtained in excellent chemical yields and up to 99% ee with less than 10% catalyst.

### GOVERNMENT INTERESTS

The claimed invention was made with Government support under grant numbers MBRS GM 08216 and NIH-IMBRE NC P20 RR-016470 awarded by the National Institutes of Health (NIH). The Government has certain rights in this invention.

### BACKGROUND

Asymmetric reduction of prochiral ketones to obtain enantiomerically pure alcohols is of one of the most important transformations in organic synthesis. In the last 25 year, a large variety of asymmetric catalysts prepared by the reaction alumino- and borohydrides with chiral diols or amino alcohols have been developed for the enantioselective carbonyl reduction with great success. In particular, the 1,2,3-oxazaborolidines derived from chiral amino alcohols have been recognized as exceptional catalysts in the reduction of aromatic ketones and in other enantioselective reactions. B-H oxazaborolidines are prepared by the reaction of the corresponding amino alcohol with borane-THF or borane dimethyl sulphide complex. Due to the extreme sensitivity of these reagents to air-moisture, they are difficult to isolate and purify, consequently, they are normally prepared in situ for subsequent reactions. However, B-H oxazaborolidines can form dimers or other species that can alter the true nature of the catalyst. In addition, other impurities present in the reaction mixture cause a detrimental effect on the enantiomeric purity of desired products and, often, the reported data is not reproducible by others. On the other hand, B-substituted oxazaborolidines, show excellent enantioselectivity and synthetic utility but, require careful purification steps to eliminate traces of boronic acid and boronic esters. Therefore, the prices of commercially available reagents are excessively high. More recently, a new enantioselective reducing reagent system has shown good to high enantioselectivities. As shown in FIG. 1, the chiral catalyst is a tight amino-borane complex 1 and borane is the external hydride donor to reduce hindered and substituted aralkyl ketones.

Additional background is provided by the following references, each of which are incorporated by reference in their entirety:
(1) Seyden-Penne, J. Reductions by the Alumino-and Borohydrides in Organic Synthesis; Wiley—VCH: New York, 1997.
(2) Daverio, P.; Zanda, M. Tetrahedron Asymm. 2001, 12, 2225-2259.
(3) Glushkov, V. A.; Tolstikov, A. G. Russ. Chem. Rev. 2004, 73, 581-608.
(4) Corey, E. J.; Helal, C. J. Angew. Chem. Int. Ed. 1998, 37, 1986-2012.
(5) Lang, A.; Noth, H.; Schmidt, M. Chem. Ber. 1996, 130, 241-246.
(6) Ortiz-Marciales, M.; De Jesús, M.; González, E.; Raptis, R. G.; Baran, P. Acta Cryst 2004, C60, 173-175.
(7) Mathre, D. J.; Thompson, A. S.; Douglas, A. W.; Hoogsteen, K.; Carroll, J. D.; Corley, E. G.; Grabowski, E. J. J. J. Org. Chem. 1993, 58, 2880-2888.
(8) Berenguer, R.; Garcia, J.; Vilarrasa, J. Tetrahedron Assymm. 1994, 5, 165-168.
(9) Jones, S.; Atherton, J. C. C. Tetrahedron Asymm. 2000, 11, 4543-4548.
(10) Kanth, J. V. B.; Brown, H. C. Tetrahedron 2002, 58, 1069-1070.
(11) Matteson, D. S. Stereodirected Synthesis with Organoboranes; Springer-Verlag: Berlin, 1995.
(12) Thormeier, S.; Carboni, B.; Kaufmann, D. E. J. Organomet. Chem. 2002, 657, 136-145.
(13) Santiesteban, F.; Campos, M. A.; Morales, H.; Contreras, R. Polyhedron 1984, 3, 589-594.
(14) Huskens, J.; Reetz, M. T. Eur. J. Org. Chem. 1999, 1775-1786.
(15) Liu, D.; Shan, Z.; Zhou, Y.; Wu, X.; Qin, J. Helvetica Chim. Act. 2004, 87, 2310-2317.
(16) Shan, Z.; Zhou, Y.; Liu, D.; Ha, W. Synthesis and Reactivity in Inorg., Metal-Org. and Nano-Metal Chem. 2005, 35, 275-279.
(17) Alexakis, A., Mutti, S. And Mangeney, P. J. Org. Chem. 1994, 59, 3326-3334.
(18) Alexakis, A., Mutti, S. And Mangeney, P. J. Org. Chem. 1992, 57, 1224-1237.

### BRIEF DESCRIPTION OF THE DRAWINGS

FIG. 1 is a schematic diagram showing the reduction of an aralkyl ketone using amino-borane complex 1 as a catalyst.

FIG. 2 is a schematic diagram showing the reduction of acetophenone using the spiroborate 2 as a catalyst.

FIG. 3 is a schematic diagram showing 12 spiroborate esters derived from chiral amino alcohols.

FIG. 4 is a schematic diagram showing the synthesis of 1-[([1,3,2]Dioxaborolan-2-yloxy)-diphenyl-methyl]-2-methylpropylamine.

FIG. 5 is a schematic diagram showing the preparation of (1R,2S)-1-(1′,3′,2′-dioxaborolan-2′-yloxy)-1-phenylpropan-2-amine.

FIG. 6 is a schematic diagram of (1R,2S)-1-(1′,3′,2′-dioxaborolan-2′-yloxy)-1-phenylpropan-2-amine.

FIG. 7 is a schematic diagram showing the preparation of (R)-(+)-2-(1,3,2-dioxaborolan-2-yloxy)-1,2,2-triphenylethanamine.

FIG. 8 is a schematic diagram showing (R)-(+)-2-(1,3,2-dioxaborolan-2-yloxy)-1,2,2-triphenylethanamine.

FIG. 9 is a schematic diagram showing five ketones.

FIG. 10 is a schematic diagram showing the general form of an enantioselective reduction reaction of a ketone using a spiroborate as a catalyst.

FIG. 11 is a schematic diagram of two amino borate ester complexes 12 and 13 suitable for use as a catalyst in an enantioselective reduction of a ketone.

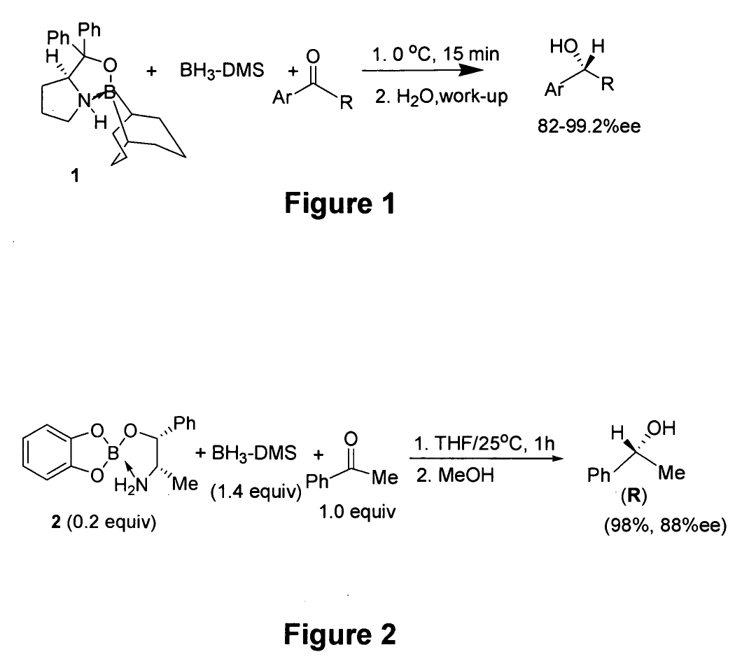

### DETAILED DESCRIPTION OF THE INVENTION

The borate 2, shown in FIG. 2, was prepared by the addition of (1R, 2S)-(−)-norephedrine to catecholborane at 0° C. for 1 hour in ether. The crystalline white solid was washed with ether and isolated with 83% yield. The yield of the reaction carried out at −78° C. was similar. By ¹¹B-NMR, it was observed the characteristic signal of the central boron atom at δ 11.6 ppm. Other signals at δ 14.2 and 7.9 ppm, were also observed indicating about 20% of impurities due to side reactions. Attempts to recrystallize the sample did not improve the purity. The reaction in THF and dichloromethane gave lower chemical yields, 42% and 46% respectively, with lower purity. The borane reduction of acetophenone was carried out in the presence of 20% equivalents of 2, obtaining the (R)-1-phenyl ethanol in quantitative yield and with 88% ee, as indicated in FIG. 2. Following these results, other solvents and aromatic ketones were enantioselectively reduced with the spiroborate 2, which was derived from catecholborane and norephedrine. The results are presented in Table 1. Higher enantioselectivities were achieved in THF and, in toluene was the lowest for 4-chloroacetophenone. The catalytic load can be lower at 10% with almost equal enantiomeric excess for acetophenone and 4-chloroacetophenone, 87, 88% ee, respectively.

TABLE 1

| Entry | Ketone | 2 | Solvent | Yield % | ee % |
|---|---|---|---|---|---|
| 1 | Acetophenone | 0.2 | THF | 98 | 88 |
| 2 | Acetophenone | 0.2 | CH₂Cl₂ | 79 | 60 |
| 3 | Acetophenone | 0.2 | dioxane | 72 | 86 |
| 4 | Acetophenone | 0.1 | THF | 53 | 87 |
| 5 | 4-Cloro- | 0.2 | THF | 74 | 87 |
| | acetophenone | | | | |
| 6 | 4-Cloro- | 0.2 | Toluene | 44 | 60 |
| | acetophenone | | | | |
| 7 | 4-Cloro- | 0.1 | THF | 82 | 88 |
| | acetophenone | | | | |
| 8 | 4-methoxy-1- | 0.2 | THF | 70 | 85 |
| | tetralone | | | | |
| 9 | 3-acetylpyridine | 0.2 | THF | 90 | 82 |
| 10 | 4-acetylpyridine | 0.2 | THF | 60 | 59 |

The yield shown in Table 1 was of product purified by Kugelrohr distillation. For entries 2, 3 and 7, the yield shown is for the crude product. The enantioselectivity was determined by GC on a chiral column (CP-Chiralsil-DexCB). For entries 9 and 10, two equivalents of borane and a 24 hour work up with methanol were required. Column 2 is the molar fraction of catalyst 2.

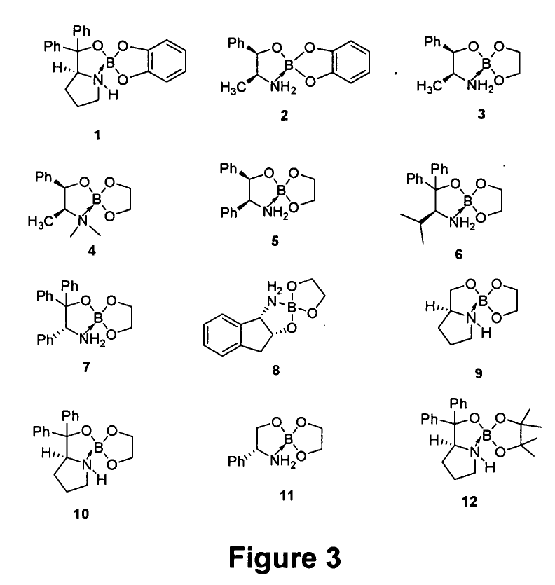

To increase the enantioselectivity, a modification of the catalytic system was made. Specifically, the structure of the chiral spiroborate ester was changed to a less strained ring system. A series of new reagents 3-12, shown in FIG. 3, were prepared from ethylene glycol, triisopropoxyborate and readily available enantiopure amino alcohols by a modification of the method reported by Huskens, J.; Reetz, M. T. Eur. J. Org. Chem. 1999, 1775-1786 (identified as reference 14 above). Specifically, the method was modified by initially heating the ethylene glycol and triisopropoxyborate before addition of the enantiopure amino alcohols. The borate esters were obtained in almost quantitatively yields with only minor impurities (table 2). The white crystalline borate amino complexes are relatively stable toward air-moisture, easy to handle under nitrogent, and no significant decomposition was observed after standing for a long period of time (4-6 months). Reagent 8 was slightly susceptible to light.

TABLE 2
Representative properties of spiroborates 3-10

| Cat. | Mp ° C. | [α]_{D}²⁰ | | ¹¹B NMR δ(ppm) |
|---|---|---|---|---|
| 3 | 176-179 | −37.5 | (C = 0.056, DMSO) | 10.0 (s) (d⁶-DMSO) |
| 4 | 70-75 | −5.0 | (C = 0.024, CHCl₃) | 10.9 (s) and 6.3 (s) |
| | | | | (CDCl₃) |
| 5 | 183 (dec) | +5.0 | (C = 0.029, DMSO) | 10.5 (bs) (d⁶⁻DMSO) |
| 6 | 207-209 | +98 | (C = 0.05, CHCl₃) | 9.6 (bs) (CHCl₃) |
| 7 | 194 (dec) | +43 | (C = 0.023 DMSO | 10.5 (s) (d⁶-DMSO) |
| 8 | 151 (dec) | +28.6 | (C = 0.018, CHCl₃) | 10.3 (s) (CHCl₃) |
| 9 | 115-116 | +13 | (C = .049, CHCl₃) | 9.9 (s) (CHCl₃) |
| 10 | 261-262 | −96.0 | (C = 0.025, CHCl₃) | 10.3 (s) (d⁶⁻DMSO) |

The first column identifies the catalyst (as shown in FIG. 3). The second column lists the melting point for each catalyst. The third column lists specific rotation. And the fourth column lists characteristic boron NMR signal.

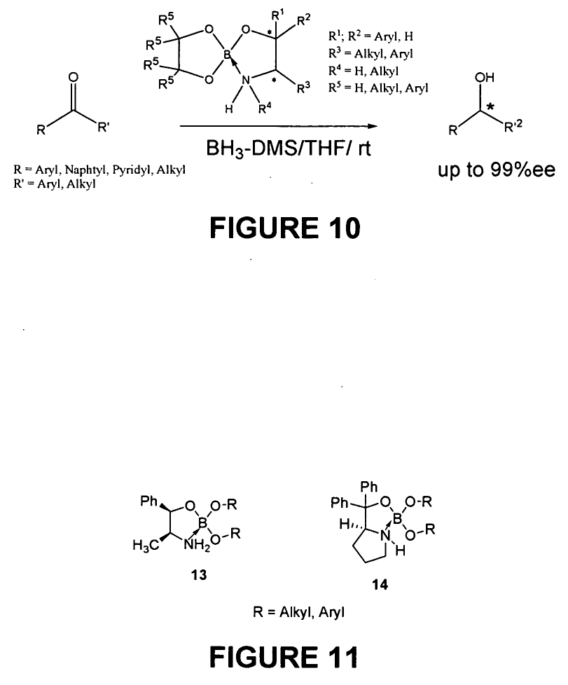

To assess the enantioselectivity of the prepared chiral spiroborates 3-10 for the reduction of aromatic ketones, acetophenone was used as a model compound. The reduction was carried out varying the amounts of catalyst with one molar equivalent of borane-DMS complex at room temperature in THF. During reduction of the acetophenone using catalyst 3, two aliquots of the reaction mixture were taken and quenched with methanol followed by water. After extraction with diethyl ether the samples were analyzed by chiral GC. The analysis showed that the reaction was complete within 15 min after entire addition of the substrate. Except for compound 4 that had lower reactivity and produced a racemic alcohol, excellent enantioselectivities were achieved with up to 10% mol of catalyst, in particular for the catalysts 6, 7 and 10. In all cases, the reactions took place with excellent reproducibility. The expensive enantio pure amino alcohols can also quantitatively recover. Table 3 summarizes the results of the enantioselective borane reduction of acetophenone with spiroborates 3-10 used as catalysts. The general form of the reduction reaction is shown in FIG. 10.

TABLE 3

| Entry | Catalyst | Mol % | Yield % | ee % | Conf. |
|---|---|---|---|---|---|
| 1 | 3 | 10 | 75 | 90 | R |
| 2 | 3 | 5 | 84 | 88 | R |
| 3 | 3 | 2.5 | 85 | 75 | R |
| 4 | 4 | 10 | 85 | 0 | — |
| 5 | 5 | 10 | 87 | 90 | R |
| 6 | 6 | 20 | 75 | 98 | R |
| 7 | 6 | 10 | 99 | 96 | R |
| 8 | 7 | 10 | 89 | 98 | S |
| 9 | 7 | 5 | 97 | 98 | S |
| 10 | 8 | 10 | 75 | 95 | S |
| 11 | 8 | 5 | 96 | 94 | S |
| 12 | 9 | 10 | 93 | 83 | R |
| 13 | 10 | 10 | 80 | 99 | R |

The first column lists the entry or reaction number. The second column identifies the catalyst used in that reaction. The third column identifies the mol percent of catalyst used. The fourth column lists the percent yield from the reaction. The product yield was purified by Kugelrohr distillation. The fifth column lists the enantioselectivity as a percentage of the yield. This is determined by GC on a chiral column (CP-Chiralsil-DexCB). For entries 1 and 4, the percentage yield is for crude product. For entry 4 traces of ketone were left after three hours. The last column identifies the chirality of the product as R or S.

The spiroborate 10 derived from (−)-α,α-diphenylpyrrolidinemethanol was particularly stable and compared in enantioselectivity to the B-substituted oxazaborolidines, offering an excellent alternative for asymmetric synthesis.

Synthesis of Catalysts:

### EXAMPLE 1

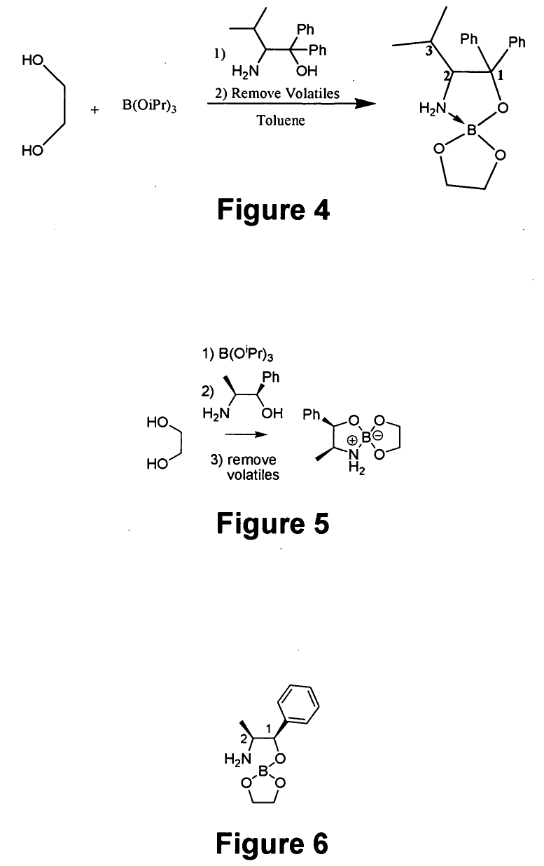

The synthesis of 1-[([1,3,2]Dioxaborolan-2-yloxy)-diphenyl-methyl]-2-methylpropylamine (Rx 165a) is shown in FIG. 4. This is also shown as catalyst 6 in FIG. 3. To a 50 mL round flask equipped with a septa and Nitrogen flow, dry ethylene glycol (0.31 g, 5.0 mmol) was added. Then dry toluene (10 mL) was added following by triisopropyl borate (1.17 mL, 5.1 mmol). The reaction mixture was gently heated to reflux until a homogeneous colorless solution was formed. A solution of Diphenyl Valinol (1.276 g, 5.0 mmol) in dry toluene (10 mL) was added to the reaction mixture while a white precipitate was observed during the process. The resulting solution with the white solid was concentrated in a rotovaporator by heating at 80° C./20 mmHg for about 1 hour until all volatiles were evaporated. The white crystalline solid was dried overnight using high vacuum to remove toluene traces. The reaction yielded 100% crude (1.620 g).

An analysis of the product gave the following results:

¹H-NMR (400 MHz, CDCl₃): δ 0.29 (d, J=6.4, 3H, CH₃), 0.78 (d, J=6.8, 3H, CH₃), 2.07 (s, 1H, C3—H), 3.83 (m, 4H, CH₂), 3.90 (s, 1H, C2—H), 4.72 (s, 2H, NH₂), 7.49-7.19 (m, 10H, Aromatic)

¹³C-NMR (100 MHz, CDCl₃): δ 16.2 (CH₃), 21.9 (CH₃), 27.4 (C3), 64.0 (C2), 65.8 (CH₂), 83.7 (C1), 126.7, 127.0, 127.2, 127.5, 127.9, 128.1, 143.4, 146.9 (Aromatic)

¹¹B-NMR (128 MHz, CDCl₃): δ 9.59 (bs)

Specific Rotation or [α]²⁰_{D}=+98 (C=0.05, CHCl₃)

Melting point: 207°-209° C.

### EXAMPLE 2

The preparation of (1R,2S)-1-(1′,3′,2′-dioxaborolan-2′-yloxy)-1-phenylpropan-2-amine is shown in FIG. 5. This is also shown as catalyst 3 in FIG. 3 To a 50-mL round bottom flask equipped with septa dry ethylene glycol (0.62 g, 10.00 mmol) was placed and dry toluene (20 mL) was added under nitrogen following by neat triisopropoxy borate (2.3 mL, 10.01 mmol). The reaction mixture was gently heated to reflux for 3 min and was cooled to room temperature after a homogeneous colorless solution was formed. Solid (1R,2S)-norephedrine (1.51 g, 10.00 mmol) was added by one portion and the resulting solution was concentrated on a rotor evaporator with heating to 80° C. for 1 hour. After all volatiles were evaporated a white crystalline residue was dried overnight using high vacuum oil pump in order to remove traces of toluene. The prepared product was analyzed by NMR and stored under nitrogen. The compound was obtained with quantitative yield (100% yield) and used for subsequent reduction reactions without other purification. The compound is also shown in FIG. 6.

An analysis of the product gave the following results:

¹H NMR (d⁶-DMSO): 7.36-7.27 (m, 4H, Hm and Ho); 7.23-7.18 (m, 1H, Hp); 5.75 (br.s, 2H, NH₂); 4.86 (d, 1H, J=5.4 Hz, C1—H); 3.67 (s, 4H, CH₂); 3.45 (br.tr, 1H, J=5.7 Hz, C2—H); 0.67 (d, 3H, J=6.8 Hz, Me).

¹³C NMR (d⁶-DMSO): 141.3 (C-i); 127.6 (C-m); 126.6 (C-p); 126.1 (C-o); 76.4 (c-1); 63.3 (CH₂); 51.7 (C-2); 13.9 (Me).

¹¹B NMR (d⁶-DMSO): 10.0 (s).

IR: 3061; 1622 (N—H); 1349; 1138; 1091; 1055.

Specific Rotation or [α]_{D}=−37.5, c=0.056 g/mL in DMSO

Melting point: 176-179° C. (dec)

### EXAMPLE 3

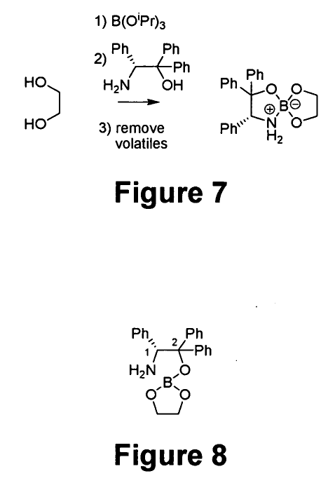

The preparation of (R)-(+)-2-(1,3,2-dioxaborolan-2-yloxy)-1,2,2-triphenylethanamine is shown in FIG. 7. This is also shown as catalyst 7 in FIG. 3 To a 50-mL round bottom flask equipped with septa dry ethylene glycol (0.62 g, 10.00 mmol) was placed and dry toluene (10 mL) was added under nitrogen following by neat triisopropoxy borate (2.3 mL, 10.01 mmol). The reaction mixture was gently heated to reflux for 3 min and was cooled to room temperature after a homogeneous colorless solution was formed. Warm solution of (R)-(+)-2-amino-1,1,2-triphenylethanol (2.89 g, 10.00 mmol) in dry toluene (15 mL) was added fast and the resulting mixture was concentrated on a rotor evaporator with heating to 80° C. for 1 hour. After all volatiles were evaporated a white crystalline residue was dried overnight using high vacuum oil pump in order to remove traces of toluene. The prepared product was analyzed by NMR and stored under nitrogen. The resulting compound was obtained with quantitative yield and used for subsequent reduction reactions without other purifications. It is shown in FIG. 8.

An analysis of the product gave the following results:

¹H NMR (d⁶-DMSO): 7.81 (d, 2H, J=7.4 Hz, H-arom); 7.50-7.45 (m, 2H, H-arom); 7.35-7.29 (m, 2H, H-arom); 7.27-7.18 (m, 3H, H-arom); 7.15-7.08 (m, 3H, H-arom); 7.01-6.88 (m, 3H, H-arom); 6.26 (br.s, 2H, NH₂); 5.18 (tr, 1H, J=5.0 Hz, C1—H); 3.85-3.60 (m, 4H, OCH₂.

¹³C NMR(d⁶-DMSO): 147.4; 145.0; 129.5; 127.4; 127.5; 127.2; 127.0; 126.8; 126.7; 126.3; 125.4; 84.8 (C2); 63.8 (C1); 63.3 (CH₂)

¹¹B NMR (d⁶-DMSO): 10.5 (s)

IR (KBr): 3272; 2908; 1584 (N—H); 1354; 1225; 1120; 1025

[α]_{D}: +43, c=0.023 g/mL in DMSO

Melting point: 194° C. (dec).

### EXAMPLE 4

The preparation of 2-[(1,3,2-dioxaborolan-2-yloxy)diphenylmethyl]pyrrolidine (catalyst 10) is as follows. To a 50 mL round flask equipped with a septa and Nitrogen flow, dry ethylene glycol (0.31 g, 5.0 mmol) was added. Then, dry toluene (15 mL) was added following by triisopropyl borate (1.17 mL, 5.1 mmol). The reaction mixture was gently heated to reflux until a homogeneous colorless solution was formed. A solution of (S)-(−)-α,α-diphenyl-2-pyrrolidinemethanol (1.267 g, 5.0 mmol) in dry toluene (10 mL) was added to the reaction mixture while a white precipitate was observed during the addition. The resulting solution with the white solid was concentrated in the rotovaporator by heating at 80° C./20 mmHg for about 1 hour until all volatiles were evaporated. The white crystalline solid was dried overnight using high vacuum to remove toluene traces. A compound was obtained with quantitative yield (1.616 g).

An analysis of the product gave the following results:

¹H-AMR (400 MHz, DMSO-d⁶): δ 1.305 (m, 1H, C4—H), 1.614 (m, 1H, C4—H), 1.664 (m, 1H, C3—H), 1.805 (m, 1H, C3—H), 2.915 (m, 1H, C5—H), 3.068 (m, 1H, C5—H), 3.592 (m, 2H, CH₂), 3.743 (m, 2H, CH₂), 4.548 (m, 1H, C2—H), 6.704 (t, 1H, NH), 7.075-7.273 (m, Aromatic), 7.526 (d, J=7.2 Hz, 2H, Ar), 7.726 (d, J=7.6 Hz, 2H, Ar).

¹³C-NMR (100 MHz, DMSO-d⁶): δ 24.30, 28.39, 45.69, 63.67 (CH₂), 63.87 (CH₂), 68.16, 81.16, 125.88, 126.76, 127.96, 127.97, 147.62 (Ar), 148.24 (Ar).

¹¹B-NMR (128 MHz, DMSO-d⁶): δ +10.34 (s).

[α]²⁰_{D}=−96.0 (c 0.025, CHCl₃).

Melting point: 261° C.-262° C.

Reductions of Ketones to Alcohols:

Spiroborate esters shown in FIG. 3 were used to reduce arylalkyl ketones, acetylpyridines and other heteroaromatic ketones to synthesize biologically active enantiopure alcohols. Table 4 summarizes the results from eight example enantioselective reductions of 3-acetylpyridine with spiroborate esters 3, 6, 7, 8 and 10 as catalysts.

TABLE 4

| Entry | Substrate | Cat. | Mol % | Yield % | Ee % | Conf. |
|---|---|---|---|---|---|---|
| 1 | 3-acetylpyridine | 3 | 20 | 84 | 91 | R(+) |
| 2 | 3-acetylpyridine | 3 | 10 | 89 | 91 | R(+) |
| 3 | 3-acetylpyridine | 3 | 5 | 94 | 90 | R(+) |
| 4 | 3-acetylpyridine | 7 | 10 | 85 | 96 | R(+) |
| 5 | 3-acetylpyridine | 6 | 10 | 94 | 96 | R(+) |
| 6 | 3-acetylpyridine | 8 | 10 | 88 | 92 | S(−) |
| 7 | 3-acetylpyridine | 10 | 10 | 80 | 99 | R(+) |
| 8 | 3-acetylpyridine | 10 | 5 | 93 | 99 | R(+) |

The first column is the entry or reaction number. The second column identifies the substrate or reactant. The third column identifies the catalyst (see FIG. 3). The fourth column lists the concentration of catalyst used (in % moles). The fifth column lists the percent yield. The sixth column lists the enantioselectivity. For entry 4, this was determined by GC on a chiral column (CP-Chiralsil-DexCB). For entry 5, this was determined by ³¹P-NMR of derivative with a phosphonate (CDA). For the other entries, this was determined by GC on a chiral column of O-acetyl derivatives. The seventh column lists the predominant chirality.

Table 5a lists the results from the enantioselective reduction of various representative aromatic and alkyl ketones with 10% spiroborates esters 6 and 10 as catalyst.

TABLE 5a

| Entry | Substrate | Cat. | Yield % | ee % |
|---|---|---|---|---|
| 1 | 4-phenylbutan-2-one | 10 | 90 | 72 |
| 2 | 1-indanone | 6 | 94 | 97 |
| 3 | 1-indanone | 10 | 96 | 96 |
| 4 | 1-tetralone | 10 | 99 | 100 |
| 5 | cyclohexyl phenyl ketone | 10 | 83 | 60 |
| 6 | p-chloroacetophenone | 10 | 98 | 99 |
| 7 | 3-chloropropiophenone | 10 | 86 | 94 |
| 8 | 2-chloro-2′,4′-difluoroacetophenone | 10 | 88 | 98 |
| 9 | 2,2,2-trifluoro-acetophenone | 10 | 97 | 82 |
| 10 | 1-adamantyl methyl ketone | 10 | 98 | 99 |

The first column lists the entry or reaction number. The second column identifies the ketone. The third column identifies the catalyst. The fourth column lists the yield. The last column lists the enatioselectivity. For entry 6, this was determined by GC on a chiral column (CP-Chiralsil-DexCB). For entries 1-4 and 8-10, this was determined by 31P-NMR of derivative with a phosphonate (CDA).

The correlation between amount of catalyst and stereoselectivity of the reduction of 3-acetylpyridine is shown in Table 5b, below, using varying concentrations of spiroborate 3 as the catalyst.

TABLE 5b

| Entry | Mol % | ee % |
|---|---|---|
| 1 | 20 | 91 |
| 2 | 10 | 91 |
| 3 | 5 | 90 |
| 4 | 2.5 | 89 |
| 5 | 1.0 | 87 |
| 6 | 0.5 | 84 |
| 7 | 0.25 | 79 |
| 8 | 0.1 | 65 |

The first column is the entry or reaction number. The second column is the mol percent of catalyst. The third column lists the enantioselectivity as a percentage of the yield. This is determined by GC on a chiral column (CP-Chiralsil-DexCB).

The effect of varying the catalytic load in the reduction of 3-acetylpyridine using catalyst 10 (shown in FIG. 3) is shown in Table 6. At only 1% of catalyst, the selectivity remains high.

TABLE 6

| Entry | Mol % | ee % |
|---|---|---|
| 1 | 10 | 99 |
| 2 | 5.0 | 99 |
| 3 | 2.5 | 98 |
| 4 | 1.0 | 98 |
| 5 | 0.5 | 94 |

The first column lists the entry or reaction number. The second column lists the mol percentage of catalyst. The third column lists the enantioselectivity as a percentage of the yield. This is determined by GC on a chiral column (CP-Chiralsil-DexCB).

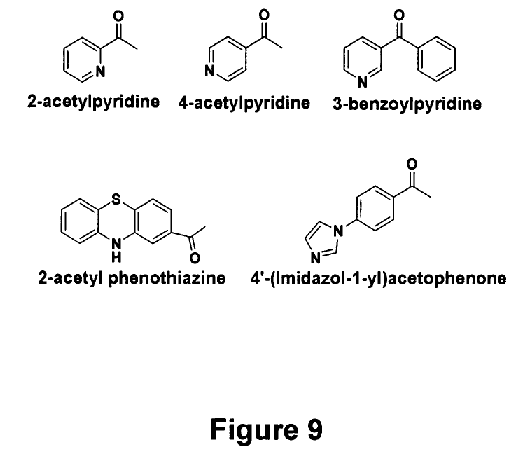

In addition, 4-acetylpyridine and other heteroaromatic compounds can be reduced using catalyst 6 and 10 (shown in FIG. 3). Ketones used in these reductions are shown in FIG. 9. The results of these reductions are summarized below in Table 7. The enantioselectivity was high even with 1% of catalyst 10. However achieving high selectivity in the reduction of 2-acetylpyridine required a stoichiometric amount of catalyst 10.

TABLE 7

| Entry | Substrate | Cat. | Mol % | Yield % | ee % |
|---|---|---|---|---|---|
| 1 | 4-acetylpyridine | 6 | 10 | 85 | 97 |
| 2 | 4-acetylpyridine | 10 | 10 | 88 | 99 |
| 3 | 4-acetylpyridine | 10 | 1 | 92 | 99 |
| 4 | 3-benzoylpyridine | 10 | 10 | 83 | 83 |
| 5 | 2-acetyl phenothiazine | 10 | 10 | 97 | 100 |
| 6 | 2-acetyl phenothiazine | 6 | 10 | 95 | 100 |
| 7 | 2-acetyl phenothiazine | 10 | 1 | — | 94 |
| 8 | 4′-(Imidazol-1-yl)acetophenone | 10 | 10 | 76 | 92 |
| 9 | 4′-(Imidazol-1-yl)acetophenone | 10 | 1 | 85 | 90 |
| 10 | 2-acetylpyridine | 10 | 100 | — | 89 |

The first column lists the entry or reaction number. The second column lists the substrate or ketone that was reduced. The third column identifies the catalyst (see FIG. 3). The fourth column lists the mol percent of catalyst used. The fifth column lists the yield percentage. The sixth column lists the percentage enantioselectivity. This is determined by ³¹P-NMR of derivative with a phosphonate (CDA), except for the last entry number 10 which was determined by GC on a chiral column of O-acetyl derivatives.

### EXAMPLE 1a

### R-(+)-α-methyl-4-pyridylmethanol

To a 100 mL round flask equipped with a septa and nitrogen flow, 10% of catalyst 6 (0.325 g, 1.0 mmol) was added. Then dry THF (30 mL) was added to make a solution. Borane complex with dimethyl sulfoxide 10.0 M (2.0 mL, 20.00 mmol) was added to the catalyst solution. The mixture was stirred for about 15 minutes. A solution of 4-acetylpyridine (1.211 g, 10.0 mmol) with dry THF (10 mL) was added to the reaction mixture during 1 hour. The reaction was allowed to react overnight. The reaction mixture was cooled to 0° C. MeOH (20 mL) was added and the mixture was heated to reflux for 4 hours. A sample of the mixture was analyzed by ¹¹B-NMR and the N—BH₃ complex signal was observed at −13.28 ppm. More MeOH (10 mL) was added and heated for 4 hours again. After decomposition of N—BH₃ complex confirmed by ¹¹B-NMR the mixture was concentrated to colorless oil. The residue was purified by column chromatography through Alumina (acid) (50 g) with an EtOAc/Hexane 1:1 mixture. The white crystalline α-methyl-4-pyridinemethanol was obtained in an 85% yield (1.048 g.) (Mp°: 55°-58° C.) According with the G.C. analysis (Column: CP-Chirasil-Dex CB, Method: Iso135) the alcohol was observed at retention time 12.39 minutes and some aminoalcohol at retention time 11.64 minutes. The enantiomeric excess of 97.2% ee was determined by ¹¹P-NMR of the phosphorus derivative.

An analysis of the product gave the following results:

¹H-NMR (400 MHz, CDCl₃): δ1.39 (d, J=6.4 Hz, 3H); 4.80 (q, J=6.4 Hz, 1H); 5.096 (s, 1H); 7.22 (d, J=6.0 Hz, 2H); 8.309 (d, J=5.6 Hz, 2H) (≈98% purity).

¹³C-NMR (100 MHz, CDCl₃): δ 24.99 (CH₃); 68.21 (C—H(OH); 120.60 (CH_{Ar}); 148.97 (CH_{Ar}); 155.97.

[α]²³_{D}=+49.0 (c=0.025, CHCl₃).

### EXAMPLE 1b

### R-(+)-α-methyl-4-pyridinemethanol

The same reduction was performed using 1% of catalyst 10 (1,3,2-dioxaborolan-2-yloxy)diphenylmethyl)pyrrolidine. Borane-DMS complex (10M, 1.6 mL, 16.00 mmol) was added to a solution of catalyst 10 (32 mg, 0.10 mmol) in dry THF (5 mL) at room temperature and the mixture was stirred for 1 hour. A solution of 4-acetylpyridine (1.21 g, 10.00 mmol) in THF (5 mL) was added for 5 h using an infusion pump. The reaction mixture was stirred at room temperature for over 1 hour, then cooled to 0° C. and quenched with methanol (10 mL). After refluxing for 12 h, the solvents were removed under vacuum, the residue was distilled (directly without chromatography purification) in a Kugelrohr apparatus under vacuum to give the final product as a white crystalline material (1.135 g, 92% yield). Chiral GC of O-acetyl derivative indicated 98.8% ee.

### EXAMPLE 1c

### R-(+)-α-methyl-4-pyrydylmethanol

The same reaction was again performed using 10% of catalyst 10 (1,3,2-dioxaborolan-2-yloxy)diphenylmethyl)pyrrolidine). Borane-DMS complex (10M, 1.7 mL, 17.00 mmol) was added to a solution of catalyst 10 (323 mg, 1.00 mmol) in dry THF (10 mL) at room temperature (during the addition hydrogen evolved) and mixture was stirred for 1 hour. A solution of 4-acetylpyridine (1.21 g, 10.00 mmol) in THF (5 mL) was added for 5 hours using an infusion pump. The reaction mixture was stirred at room temperature for over 1 hour, then cooled to 0° C. and quenched with methanol (10 mL). After refluxing for 12 hours, the solvents were removed under vacuum, the residue was distilled (directly without chromatography purification) in a Kugelrohr apparatus under vacuum to give the final product as white crystalline material (1.077 g, 88% yield). Chiral GC of O-acetyl derivative indicated 99.0% ee.

### EXAMPLE 2a

### R-(+)-α-methyl-3-pyridylmethanol

To a 100 mL round flask equipped with a septa and Nitrogen flow, 10% of catalyst 6 (0.325 g, 1.0 mmol) was added. Then dry THF (30 mL) was added to make a solution. Borane complex with dimethyl sulfoxide 10.0 M (2.0 mL, 20.00 mmol) was added to the catalyst solution. The mixture was stirred for about 15 minutes. A solution of 3-acetylpyridine (1.211 g, 10.0 mmol) with dry THF (10 mL) was added to the reaction mixture during 1 hour. The reaction was allowed to react overnight. The reaction mixture was cooled to 0° C. MeOH (30 mL) was added and the mixture was heated to reflux for 8 hours. Decomposition of N—BH₃ complex was confirmed by ¹¹B-NMR and the mixture was concentrated to colorless oil. The residue was distilled with high vacuum in the Kugelrohr oven to obtain 1.161 g (94% yield ) of (R)-(+)-α-methyl-3-pyridinemethanol. The boiling point was measured at 140° C. with 0.7 mmHg. Enantiomeric excess of 96.4% ee was determined by ³¹P-NMR.

An analysis of the product gave the following results:

¹H-NMR (400 MHz, CDCl₃): δ 1.46 (d, J=6.4 Hz, 3H, CH₃); 4.87 (q, J=6.4 Hz, 1H, *C—H); 5.92 (s, 1H, OH), 7.21 (m, 1H, C2—H); 7.72 (dt, J=8.0 Hz, 1H, C3—H); 8.28 (dd, J=5.2 Hz, 1H, C1—H); 8.40 (d, J=2.0 Hz, 1H C5—H).

¹³C-NMR (100 MHz, CDCl₃): δ 24.97; 66.91; 123.29; 133.42; 141.88; 146.59; 147.40.

[α]²³_{D}=+40.9 (c=0.031, CHCl₃).

### EXAMPLE 2b

### R-(+)-α-methyl-3-pyridylmethanol

The same reaction was performed using 1% of catalyst 10 (1,3,2-dioxaborolan-2-yloxy)diphenylmethyl)pyrrolidine). Borane-DMS complex (10M, 1.6 mL, 16.00 mmol) was added to a solution of (S)-2-((1,3,2-dioxaborolan-2-yloxy)diphenylmethyl) pyrrolidine 10 (32 mg, 0.10 mmol) in dry THF (5 mL) at room temperature (during the addition hydrogen evolved) and mixture was stirred for 1 hour. A solution of 3-acetylpyridine (1.21 g, 10.00 mmol) in THF (5 mL) was added for 5 hours using an infusion pump. The reaction mixture was stirred at room temperature for over 1 h, then cooled to 0° C. and quenched with methanol (10 mL). After refluxing for 12 hours, the solvents were removed under vacuum, the residue was distilled (directly without chromatography purification) in a Kugelrohr apparatus under vacuum to give the final product as colorless oil (1.18 g, 96%). Chiral GC of O-acetyl derivative indicated 98.2% ee.

### EXAMPLE 3a

### R-(+)-phenyl(pyridin-3-yl)methanol

To a 100 mL round flask equipped with a septa and nitrogen flow, 10% of diphenylprolinol-borate or catalyst 10 (0.323 g, 1.0 mmol) was added. Then dry THF (30 mL) was added to make a solution. Borane complex with dimethyl sulfoxide 10.0 M (1.0 mL, 10 mmol ) was added to the catalyst solution. The mixture was stirred for about 15 minutes. A solution of 3-benzoylpyridine (1.832 g, 10.0 mmol) with dry THF (10 mL) was added to the reaction mixture during 1 hour. The reaction mixture was cooled to 0° C., MeOH (20 mL) was added and the mixture was heated to reflux for 8 hours. Decomposition of N—BH₃ complex was confirmed by ¹¹B-NMR and the mixture was concentrated to colorless oil. The residue was distilled with high vacuum in the Kugelrohr oven to obtain 1.533 g (83% yield) of phenyl (pyridin-3-yl)methanol with a boiling point of 140° C. at 0.7 mmHg. Enantiomeric excess of 83.0% ee was determined by ³¹P-NMR.

An analysis of the product gave the following results:

¹H-NMR (400 MHz, CDCl₃): δ 4.203 (s, 1H, OH); 5.843 (s, 1H, C—H); 7.23 (m, 1H, C—H), 7.27-7.38 (m, 5H); 7.71 (dt, J=7.6 Hz, 1H); 8.36 (dd, J=4.4 Hz, 1H); 8.50 (d, J=2.4 Hz, 1H).

¹³C-NMR (100 MHz, CDCl₃): δ 73.90, 123.47, 126.56, 127.90, 128.70, 134.38, 139.69, 143.25, 148.04, 148.34.

[α]²³_{D}=+12.0 (C=0.016, CHCl₃).

### EXAMPLE 4

### R-(+)-Phenyl ethanol

To a 100 mL round flask equipped with a septa and Nitrogen flow, 10% of catalyst 10 (0.323 g, 1.0 mmol) was added. Then dry THF (30 mL) was added to make a solution. Borane dimethyl sulfide complex (BDS) 10.0 M (1.0 mL, 10 mmol ) was added to the catalyst solution. The mixture was stirred for about 15 minutes. A solution of acetophenone (1.201 g, 10.0 mmol) with dry THF (10 mL) was added to the reaction mixture during 1 hour. After 15 minutes the reaction was monitored by GC, indicating that acetophenone was consumed. The solution was stirred at room temperature for over 1 hour, then cooled to 0° C. and quenched with methanol (10 mL). After stirring for 1 hour at room temperature the solvents were removed under vacuum, the residue was dissolved dichloromethane (DCM) (40 mL), washed with saturated solution of ammonium chloride (25 mL), water (25 mL) and dried with sodium sulfate. The solvents were removed under vacuum and the residue was distilled in a Kugelrohr apparatus under vacuum (59° C./0.25 mmHg) to give the final product, 1-phenylethanol, as a colorless oil (1.20 g, 98% yield). Chiral GC indicated a ratio of enantiomers as 99.53: 0.47 or 99% ee.

An analysis of the product gave the following results:

¹H-NMR (400 MHz, CDCl₃): δ 1.42 (d, J=6.4 Hz, 3H, CH₃); 2.62 (d, 1H, OH); 4.79 (m, 1H, CH); 7.20-7.31 (m, 5H, Ar).

¹³C-NMR (100 MHz, CDCl₃): δ 25.00 (CH₃); 70.12 (C*—H); 125.30 (Ar); 127.25 (Ar); 128.32 (Ar); 145.75 (Ar).

[α]²⁰_{D}=+43.8 (c 0.039, MeOH).

### EXAMPLE 5

### R-(+)-1-4-tolylpropan-1-ol

To a 100 mL round flask equipped with a septa and Nitrogen flow, 10% of catalyst 6 (0.325 g, 1.0 mmol) was added. Then dry THF (30 mL) was added to make a solution. BDS complex 10.0 M (1.0 mL, 10.00 mmol) was added to the catalyst solution. The mixture was stirred for about 15 minutes. A solution of 4-methyl propiophenone (1.482 g, 10.0 mmol) with dry THF (10 mL) was added to the reaction mixture during 1 hour. The reaction was allowed to react overnight. A sample of 0.5 mL was treated with MeOH (2 mL) and water (1 mL) followed by Et₂O extractions (3 mL). The crude was analyzed by G.C. and the product was observed with a retention time of 14.643 min with an approximately enantiomeric excess of 82% ee. The reaction mixture was cooled to 0° C., MeOH (15 mL) was added and the mixture is heated in the rotovaporator while concentrated. The concentrate was treated with NH₄Cl saturated solution followed by extractions with DCM (4×25 mL), dried with sodium sulfate and concentrated. After vacuum distillation with the Kugelrohr oven (150° C./0.15 mmHg) the 1-4-tolylpropan-1-ol was obtained in an 82% yield (1.230 g).

An analysis of the product gave the following results:

¹H-NMR (400 MHz, CDCl₃): δ 0.856 (t, J=7.4 Hz, 3H, CH₃); 1.684 (m, 2H, CH₂); 2.315 (s, 3H, CH₃); 2.542 (s, 1H, OH); 4.447 (t, J=6.6 Hz, 1H, CH); 7.098, 7.118, 7.156, 7.176 (Ar, 4H).

¹³C-NMR (100 MHz, CDCl₃): δ 10.04 (CH₃); 20.94 (CH₃); 31.62 (CH₂); 75.59 (CH); 125.85, 128.85, 136.79, 141.58 (Ar); (Mass, 70 eV, EI): 150.1 (M⁺, 4.43%); 133.1 (100%), 121.1 (48.36%); 93.1 (63.43%); 91.1 (41.95%); 77.1 (13.61%).

[α]²⁰_{D}=+40.7 (C=0.063, CHCl₃).

### EXAMPLE 6a

### R-(−)-1-indanol

To a 100 mL round flask equipped with a septa and Nitrogen flow, 10% of catalyst 6 (0.325 g, 1.0 mmol) was added. Then dry THF (30 mL) was added to make a solution. 10.0 molar borane-DMS complex (1.0 mL, 10.00 mmol) was added to the catalyst solution. The mixture was stirred for about 15 minutes. A solution of 1-indanone (1.322 g, 10.0 mmol) with dry THF (10 mL) was added to the reaction mixture during 1 hour. At the end of the addition a sample of 0.5 mL was treated with MeOH (2 mL) and water (2 mL) followed by Et₂O extractions (3 mL). The crude was analyzed by G.C. and product was observed with a retention time of 15.473 minutes with an approximately enantiomeric excess of 78%. The reaction was allowed to react overnight. The reaction mixture was cooled to 0° C. MeOH (20 mL) was added and the mixture is heated in the rotovaporator while concentrated. The concentrate was treated with NH₄Cl saturated solution (25 mL) followed by extractions with DCM (4×20 mL), dried with sodium sulfate and concentrated. After vacuum distillation with the Kugelrohr oven (136° C./0.6 mmHg) the white solid of 1-indanol was obtained in a 94% yield (1.264 g). A 97.4% ee was determined by ³¹P-NMR.

An analysis of the product gave the following results:

¹H-NMR (400 MHz, CDCl₃): δ 1.87 (m, 1H, C8—H); 22.05 (s, 1H, OH). 41 (m, 1H, C8—H); 2.75 (m, 1H, C9—H); 2.99 (m, 1H, C9—H); 5.16 (t, J=6.2 Hz, 1 H, C1—H); 7.15-7.19 (m, Ar, 3H); 7.35 (d, J=5.6 Hz, 1H, Ar).

¹³C-NMR (100 MHz, CDCl₃): δ 29.72; 35.82; 76.31; 124.15; 124.82; 126.62; 128.22; 143.24; 144.94; (Mass, 70 eV, EI): 134.1 (M⁺, 46.83%); 133.1 (100%), 117.2 (75.55%); 105.1 (10.08%).

[α]²⁰_{D}: −29.4 (c 0.033, CHCl₃);

Melting point: 68°-69° C.

### EXAMPLE 6b

### R-(−)-1-indanol

Using catalyst 10 (1.0 mmol, 10%) and following a similar procedure as above in Example 6a, the R-(−)-1-indanol (2,3-dihydro-1H-inden-1-ol) was obtained as a solid (1.29 g, 96% yield).

An analysis of the product showed:

³¹P-NMR (CDCl₃) δ 144.97 ppm (98%), 138.27 ppm (2%)—96% ee.

### EXAMPLE 7

### 1-(4-chlorophenyl)-ethanol

To a 100 mL round flask equipped with a septa and Nitrogen flow, 10% of catalyst 10 (0.323 g, 1.0 mmol) was added. Then dry THF (30 mL) was added to make a solution. Borane dimethyl sulfide complex (BDS) 10.0 M (1.0 mL, 10 mmol ) was added to the catalyst solution. The mixture was stirred for about 15 minutes. A solution of 1-(4-chlorophenyl)-ethanone (1.30 mL, 10 mmol) in dry THF (15 mL) was added drop wise by a syringe using infusion pump for 1 hour. After addition was completed the reaction mixture was stirred for 2.5 hours at room temperature. The reaction mixture was cooled to 0° C. and quenched with methanol (40 mL). The addition was done using an infusion pump for 1 hour. The mixture was left stirred overnight. The solvents were removed under high vacuum and the residual was dissolved in DCM (40 mL), washed with saturated solution of ammonium chloride NH₄Cl (40 mL) twice, then water (3×15 mL), dried with Na₂SO₄, filtered and concentrated in the rotavapor. The reaction yielded 1.53 g (98% yield). GC-Chiral Column (CP-Chiralsil-Dex CB) Method: ISO140.M: 9.84 min (0.56%), 10.12 min (99.44%) 98.9% ee. Purification by flash silica column chromatography with hexane:ethyl acetate 1:1, gave 1.326 g (85%) of desired product that was analyzed by GC-Chiral Column (CP-Chiralsil-Dex CB) (Method: ISO140.M): 9.77 min (0.39%), 10.03 min (99.61%) 99.2% ee.

### EXAMPLE 8

### R-(+)-3-chloro-1-phenylpropan-1-ol

Following similar procedure as before, BH₃.SMe₂ (10 M, 0.7 mL, 7 mmol) was added to a solution of complex derived from ethylene glycol and diphenyl prolinol (EG-DDP), catalyst 10, (323 mg, 0.1 mmol) in dry THF (35 mL) at room temperature. The reaction mixture was stirred for approximately one hour. A solution of dry 3-chloropropiophenone (1.69 g, 10 mmol) in dry THF (5 mL) was added by a syringe using infusion pump for 1 hour. (Rate: 6 mL/h). The 3-chloropropiophenone solution was light yellow but after adding to the complex, the total solution was clear. Following similar procedure as above for the work-up, the crude product was obtained: 1.66 g (97% yield). The product was analyzed by ³¹P-NMR: 145.0 ppm (5.7%), 134.5 ppm (94.3%)→88.6% ee. The product was purified by column chromatography with 30 g of silica and a mobile phase of hexane/ethyl acetate (2:1).(Yield: 1.47 g, 86%). The product was analyzed by ¹H, ¹³C, and 31P-NMR (derivative with a phosphonate (CDA). ³¹P-NMR: 145.1 ppm (3%), 134.6 ppm (97%): 94% ee; [α]²⁰=+21.0 c=0.030 (CHCl₃).

Although the invention has been described with reference to specific catalyst and reactions, those skilled in the art will appreciate that many modifications can be made without departing from the scope of the invention. For one example, although the catalysts shown included a ring derived from glycol, other amino borate ester complexes could be used. Two are shown in FIG. 11. All such modifications or equivalents are intended to be encompassed within the scope of the claims.

1-32. (canceled)

33. A chiral accessory having the formula I: 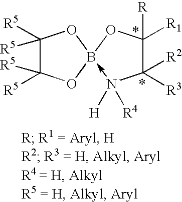
where, the R1 and R2 groups are equal or different; a hydrogen atom or a substituted or unsubstituted, aryl, alkyl, cycloalkyl or aralkyl;
R2 and R3 groups are different; a hydrogen atom or a substituted or unsubstituted, alkyl, aryl, aralkyl group; and the R4 group is a H or a cycloalkyl or aralkyl group;
wherein the substituents R, R1, R2, R3, R4 and R5 groups are substantially non-reactive.

34. The chiral accessory of claim 33, where the R and R1 groups are each an H, phenyl or aralkyl group.

35. The chiral accessory of claim 33, wherein R2 and R3 are different; a H, a methyl, substituted alkyl, phenyl or aralkyl groups, and the R4 group is a H or a cyclopentyl group.

36. The chiral accessory of claim 33, wherein said chiral accessory comprises:

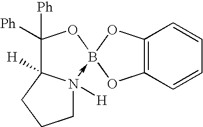

37. The chiral accessory of claim 33, wherein said chiral accessory comprises:

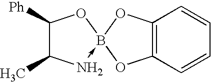

38. The chiral accessory of claim 33, wherein said chiral accessory comprises:

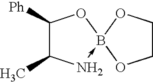

39. The chiral accessory of claim 33, wherein said chiral accessory comprises:

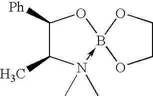

40. The chiral accessory of claim 33, wherein said chiral accessory comprises:

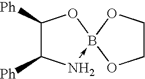

41. The chiral accessory of claim 33, wherein said chiral accessory comprises:

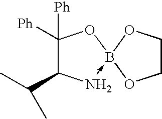

42. The chiral accessory of claim 33, wherein said chiral accessory comprises:

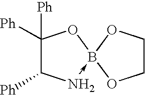

43. The chiral accessory of claim 33, wherein said chiral accessory comprises:

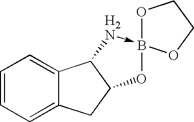

44. The chiral accessory of claim 33, wherein said chiral accessory comprises:

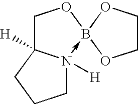

45. The chiral accessory of claim 33, wherein said chiral accessory comprises:

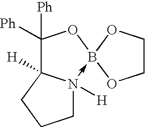

46. The chiral accessory of claim 33, wherein said chiral accessory comprises:

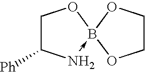

47. The chiral accessory of claim 33, wherein said chiral accessory comprises:

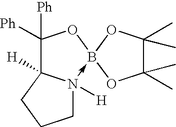

48. The chiral accessory of claim 33, wherein said chiral accessory is prepared at least one month prior to its usage on the reduction of a prochiral ketone.

49. The chiral accessory of claim 33, wherein said chiral accessory is able to reduce a prochiral ketone to obtain an enantiomeric excess of at least 97%.

50. A process for asymmetrically reducing a prochiral ketone in the presence of the chiral accessory of claim 33.

51. The process of claim 50, wherein said prochiral ketone has the formula II: 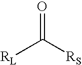
where, RL and RS are different, and each are an unsubstituted or substituted, aryl, alkyl, cycloalkyl, aralkyl, heterocyclic or heteroaryl group,
the process comprising reacting the prochiral ketone having the formula II with borane derived from a borane reagent in the presence of said chiral accesory, to form a chiral alcohol having the formula III: 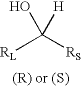
where, RL and RS are the same as defined above for the prochiral ketone having the formula II.

52. The process of claim 50, wherein the reduction requires between about 0.01 to about 0.1 equivalent of said chiral accessory.

53. The process of claim 51, where RL is the unsubstituted or substituted, aryl, aralkyl, heteroaryl, unsubstituted or substituted pyridyl groups; and RS is the unsubstituted or substituted alkyl, cycloalkyl, pyridyl or heteroaryl group.
